(19) United States
(12) Patent Application Publication
(10) Pub. No.: US 2025/0134599 A1
(43) Pub. Date: May 1, 2025

(54) UNIVERSAL TOOL ADAPTER

(71) Applicant: AUGMEDICS LTD., Yokneam Illit (IL)

(72) Inventors: Stuart Wolf, Yokneam (IL); Nissan Elimelech, Beerotaim (IL); Tomer Gera, Kfar Tavor (IL)

(21) Appl. No.: 18/988,285

(22) Filed: Dec. 19, 2024

Related U.S. Application Data
(63) Continuation of application No. 17/015,199, filed on Sep. 9, 2020, now Pat. No. 12,239,385.

(51) Int. Cl. A61B 34/20; A61B 17/00; A61B 17/88; A61B 90/00

(52) U.S. Cl. CPC A61B 34/20; A61B 17/8875; A61B 90/03; A61B 90/39; A61B 2017/00486; A61B 2034/2055; A61B 2090/031; A61B 2090/3937; A61B 2090/3983

(57) ABSTRACT

A tool adapter, consisting of an adapter arm having a proximal end terminating with a connection and a distal end having a circular coupling, the circular coupling having a center and defining an axis orthogonal to the circular coupling and passing through the center. The adapter also has a tool grip rotatingly connected to the circular coupling so as to permit rotation of the tool grip about the axis, the tool grip being configured to fixedly retain a tool along the axis.

### CROSS-REFERENCE TO RELATED APPLICATIONS

This application is a continuation of U.S. patent application Ser. No. 17/015,199, filed Sep. 9, 2020, which is incorporated herein by reference in its entirety.

### FIELD OF THE INVENTION

The present invention relates generally to surgery, and specifically to a tool adapter used to hold a tool during the surgery.

### BACKGROUND OF THE INVENTION

During surgery, it is often necessary to have a tool used in the surgery held, typically in a relatively fixed position. While the surgeon performing the surgery may hold the tool, having the tool held mechanically frees up the surgeon's hands for other tasks. A number of systems for holding a tool are known in the art.

U.S. Pat. No. 5,441,042 to Putman describes a universal positioning arm employing a clamp having quick disconnect means for releasably holding a surgical instrument such as an endoscope during a surgical procedure. The clamp includes a shaft with a bayonet connector.

U.S. Pat. No. 5,792,046 to Dobrovolny describes a surgical support structure joint which includes a shaft having an axis, a first support member rotatably supported about the axis of the shaft and a camming member coupled to the first support member. The camming member is movable between a first clamped position and a second unclamped position.

U.S. Pat. No. 6,610,009 to Person describes an apparatus for holding a surgical instrument relative to a base. The apparatus has a mounting portion configured and dimensioned to engage a portion of a base, and a jaw assembly including first and second jaw members which define a retaining area configured and dimensioned to retain the shaft of a surgical instrument.

U.S. Pat. No. 6,856,826 to Seeley et al. describes a method for surgical imaging and display including positioning a defined set of markers disposed in a pattern so as to be imaged in each pose or view of an imaging assembly, the set of markers being fixed in pre-determined positions in a rigid carrier.

U.S. Pat. No. 7,043,961 to Pandey et al. describes how a standard medical or surgical tool's axis is located by attaching to the tool a plurality of a location indicating elements or markers having a known geometric or spatial relationship, and placing the tool in a tool calibrator that supports the tool in a manner that allows tools of different diameters to be rotated while maintaining the tool's axis in a fixed or stable orientation.

U.S. Pat. No. 7,229,078 to Lechot describes a surgical tool holder which comprises a shank having a first driveable end and a second coupling end, the second end comprising a coupling device having interfaces for mating with corresponding structures on the inside of a hollow surgical tool.

U.S. Pat. No. 7,320,556 to Vagn-Erik describes a gripping device which comprises a support body within a sleeve, with the sleeve being mounted around the body for relative translational movement along a common axis.

U.S. Pat. No. 7,435,219 to Kim describes a surgical retractor positioning device that creates a surgical working field in a minimally invasive and flexible manner. In one embodiment, the device can include a frame and an arm connected to the frame.

U.S. Pat. No. 7,840,256 to Lakin et al. describes an array for use with a surgical navigation system. The array comprises a frame and first, second and third markers attached to the frame. The first, second and third markers are detectable by a tracking system used in image guided surgery, and the first marker is movable relative to the frame.

U.S. Pat. No. 7,857,271 to Lees describes a surgical tool holder that includes a holding member with a clamping jaw, which is movable between open and clamping positions. A gripping member is configured to grip a surgical tool and is selectively placed in a rotatable or fixed association with the holding member.

U.S. Pat. No. 7,881,770 to Melkent et al. describes apparatus, for use within an image-guided surgical navigation system, that facilitates the combined positioning and orientation of multiple surgical implements. A tool guide having multiple cannulas is tracked by a surgical navigation system in real time.

U.S. Pat. No. 7,993,353 to Roßner et al. describes a marker system, components and method for use with a medical navigation system, where the marker system includes interchangeable tracking marker holders and fastener bases for attaching the marker holders to instruments or body parts that are to be tracked during a medical procedure.

U.S. Pat. No. 9,149,317 to Arthur et al. describes a device for holding a surgical tool. The device comprises a base extending along a longitudinal axis between a proximal end and a distal end. The distal end is configured for engagement with a body of a patient to stabilize the device.

U.S. Pat. No. 9,179,984 to Teichman et al. describes a navigation system that includes a multi-configuration tracking array that extends from a first medical instrument. A plurality of tracking devices can be positioned on the multi-configuration tracking array.

U.S. Pat. No. 9,532,849 to Anderson et al. describes a robotic surgical system for performing a procedure within a sterile field. The system comprises a manipulator arm and a surgical accessory clamp for coupling a surgical accessory to a distal end portion of the manipulator arm. The accessory clamp includes a base for coupling with the distal end portion of the manipulator arm and two clamp jaws.

U.S. Pat. No. 9,724,165 to Arata et al. describes a surgical system which can identify an interface on an anatomy of a patient. The interface may comprise a painted portion of a bone of the patient, a divot made in the bone, or a mechanical interface that includes a portion configured to be affixed to the bone.

US Patent Application 2016/0256223 to Haimeri et al. describes a data processing method for generating compensation information for tracking or for determining the position and/or orientation of an object in space.

Documents incorporated by reference in the present patent application are to be considered an integral part of the application except that, to the extent that any terms are defined in these incorporated documents in a manner that conflicts with definitions made explicitly or implicitly in the present specification, only the definitions in the present specification should be considered.

### SUMMARY OF THE INVENTION

An embodiment of the present invention provides a tool adapter, consisting of:
an adapter arm having a proximal end terminating with a connection and a distal end having a circular coupling, the circular coupling having a center and defining an axis orthogonal to the circular coupling and passing through the center; and
a tool grip rotatingly connected to the circular coupling so as to permit rotation of the tool grip about the axis, the tool grip being configured to fixedly retain a tool along the axis.

In a disclosed embodiment the tool grip includes a collet housed in a collet holder, and the collet is configured on compression to grip the tool. The collet holder may be held by the circular opening, and may be configured to be unable to translate along the axis. The tool adapter may include a collet fastening head configured to screw onto the collet holder so as implement the compression of the collet.

In a further disclosed embodiment the tool adapter includes a position marker fixedly connected to the connection of the proximal end in a preset spatial relationship with the axis, the marker having optical elements enabling the marker to be tracked spatially, so that tracking of the marker provides tracking of the tool retained by the tool grip.

In a yet further disclosed embodiment the tool adapter includes a torque limiting device coupled to the tool grip and configured to apply a preset threshold torque to the grip so that rotation of the tool grip is permitted when the threshold torque is exceeded. The torque limiting device may consist of a plurality of pins retained within the tool grip and configured to exert force against the circular opening.

In an additional disclosed embodiment the tool adapter includes a total indicated runout (TIR) controller coupled to the tool grip and configured to apply a pre-determined TIR to the tool.

There is further provided, according to an embodiment of the present invention, a method for conducting image guided surgery, consisting of:
providing an adapter arm having a proximal end terminating with a connection and a distal end having a circular coupling, the circular coupling having a center and defining an axis orthogonal to the circular coupling and passing through the center;
rotatingly connecting a tool grip to the circular coupling so as to permit rotation of the tool grip about the axis, the tool grip being configured to fixedly retain a tool along the axis;
fixedly connecting a position marker to the connection of the proximal end of the adapter arm in a preset spatial relationship with the axis, the position marker including optical elements enabling the marker to be tracked spatially, so that tracking of the position marker provides tracking of the tool fixed to the enclosure; and
presenting an image of the tracked tool to a professional performing the surgery.

The present disclosure will be more fully understood from the following detailed description of the embodiments thereof, taken together with the drawings, in which:

### BRIEF DESCRIPTION OF THE DRAWINGS

FIG. 1 schematically illustrates use of a tool adapter in an augmented reality system, according to an embodiment of the present invention;

FIG. 2 is a schematic diagram illustrating an assembly, according to an embodiment of the present invention;

FIG. 3 is a schematic diagram illustrating a tool adapter and a tool, according to an embodiment of the present invention;

FIG. 4 is a schematic exploded view of the tool adapter and the tool, according to an embodiment of the present invention;

FIG. 5 is a schematic cross-section of the tool adapter and the tool, according to an embodiment of the present invention;

FIG. 6 is a schematic diagram illustrating a tool adapter and a tool, according to an alternative embodiment of the present invention; and

FIG. 7 is a flowchart of steps performed in using a tool adapter in the augmented reality system, according to an embodiment of the present invention.

### DETAILED DESCRIPTION OF EMBODIMENTS

### Overview

During a medical procedure performed using an augmented reality system, a tool used in the procedure, such as a screwdriver used for pedicle screws, typically needs to be tracked, so that images presented to a medical professional using the system correctly register with the tool and with the patient undergoing the procedure. In order to track the tool, a marker is typically connected to the tool, and the marker is tracked by the system. However, in the case of tools such as screwdrivers that need one or more rotations, or even a partial rotation, the marker must stay within the field of view of the tracking system so that tracking is maintained. Furthermore, to eliminate tracking errors, total indicated runout should be minimized.

Embodiments of the present invention provide a solution that overcomes both of these problems by attaching a tool adapter having the marker to the tool, and configuring the tool adapter to be able to rotate about the tool without changing the spatial relationship of the tool to the marker.

The tool adapter comprises an adapter arm which terminates at a proximal end in a connection to the marker, and at a distal end in a circular opening. A tool grip is rotatingly connected to the circular opening so as to permit rotation of the tool grip about an axis defined by the opening, the tool grip being configured to fixedly retain a tool along the axis.

The tool grip typically comprises a collet housed in a collet holder, and the collet is configured to grip the tool when the collet is compressed. Use of a collet firmly and stably holds the tool in a centered location. The collet holder is held in the circular opening, and the combination of the collet, collet holder, and circular opening means that the shaft of the tool remains accurately along the axis of the opening during rotation, and that there is very low total indicated runout (TIR) during all rotations. The inventors have found that in contrast to prior art systems that have a TIR of the order of 100 microns, embodiments of the present invention have a TIR of the order of 10 microns, and thus substantially improve the accuracy of tracking of a gripped tool.

### System Description

In the following, all directional references (e.g., upper, lower, upward, downward, left, right, top, bottom, above, below, vertical, and horizontal) are only used for identification purposes to aid the reader's understanding of the present invention, and do not create limitations, particularly as to the position, orientation, or use of embodiments of the invention.

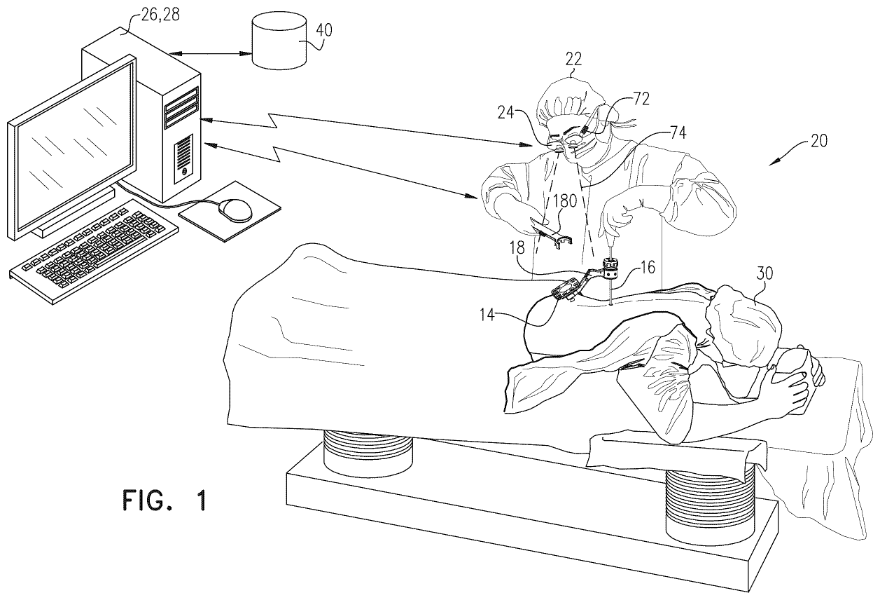

Reference is now made to FIG. 1, which schematically illustrates use of a tool adapter 18 in an augmented reality system 20, according to an embodiment of the present invention. By way of example and for simplicity, in the following description system 20 is assumed to be used by a medical professional 22 in a medical procedure wherein adapter 18 is attached to a cylindrical tool 16, for example a screwdriver, using a wrench 180, as is explained in more detail below. While the description herein assumes a medical procedure, it will be understood that embodiments of the present invention may be used in non-medical situations.

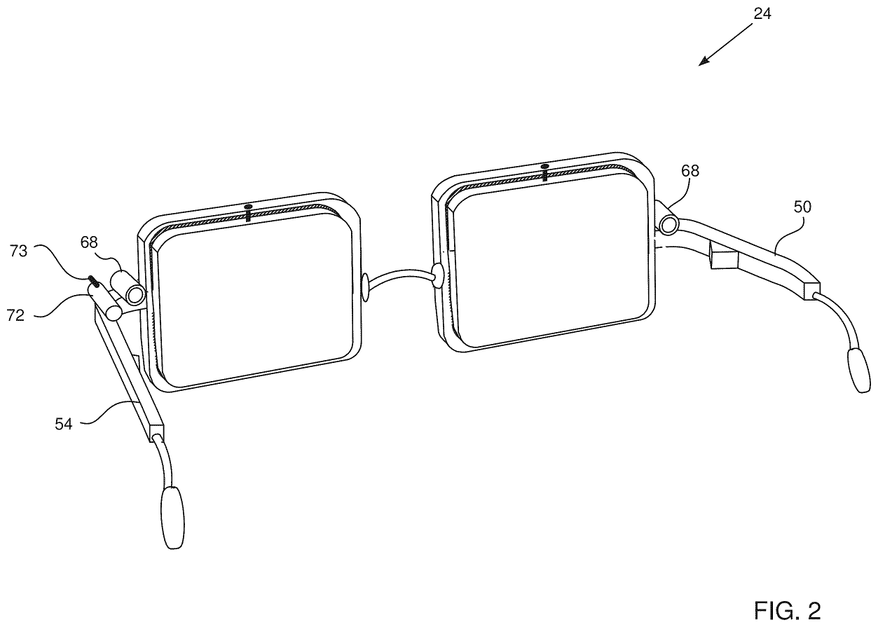

System 20 is operated by medical professional 22, who wears, by way of example, an augmented reality assembly 24, described in more detail below with respect to FIG. 2, which tracks tool adapter 18. It will be understood that assembly 24 is but one type of tracking system that is able to track the tool holder, and that the scope of the present invention comprises any tracking system able to track tool adapter 18.

Assembly 24 comprises, inter alia, an image capturing device 72, also termed herein a camera 72, that has a field of view 74 and that is configured to capture images in the visible spectrum. Assembly 24 and functions of system 20, processor 26, and device 72 are described below. An assembly similar to augmented reality assembly 24, and its operation, are described in U.S. Pat. No. 9,928,629, to Benishti, et al., whose disclosure is incorporated herein by reference.

While assembly 24 may be incorporated for wearing into a number of different retaining structures on professional 22, in the present description the retaining structure is assumed to be similar to a pair of spectacles. Those having ordinary skill in the augmented reality art will be aware of other possible structures, such as incorporation of the augmented reality assembly into a head-up display that is integrated into a headset worn by the user of system 20, and all such structures are assumed to be comprised within the scope of the present invention.

System 20 comprises and is under overall control of a processor 26. In one embodiment processor 26 is assumed to be incorporated within a stand-alone computer 28, and the processor typically communicates with other elements of the system, including assembly 24, wirelessly, as is illustrated in FIG. 1. Alternatively or additionally, processor 26 may use optical and/or conducting cables for communication. In further alternative embodiments processor 26 is integrated within assembly 24, or in the mounting of the assembly. Processor 26 is typically able to access a database 40, wherein are stored images and other visual elements used by system 20. Software enabling processor 26 to operate system 20 may be downloaded to the processor in electronic form, over a network, for example. Alternatively or additionally, the software may be provided on non-transitory tangible media, such as optical, magnetic, or electronic storage media.

The medical procedure exemplified here is on a patient 30, and during the procedure a position marker 14 is incorporated into tool adapter 18. As described below, marker 14 is trackable by processor 26, so that the processor is able to track also the tool adapter to which the marker is attached, and a tool 16 held by the adapter.

FIG. 2 is a schematic diagram illustrating assembly 24, according to an embodiment of the present invention. As stated above, assembly 24 is configured, by way of example, as a pair of spectacles 50 mounted on a frame 54.

At least one image capturing device 68 is attached to frame 54. Typically, devices 68 comprise cameras configured to capture images of scenes viewed by the professional's eyes, including images of marker 14 in the visible spectrum.

As stated above assembly 24 also comprises camera 72, which is configured to capture images of elements of a scene, including marker 14, in front of assembly 24. The images are produced from radiation projected by a projector 73 that is in the spectrum detected by camera 72. Projector 73 is located in close proximity to camera 72, so that radiation from the projector, that has been retroreflected, is captured by camera 72. The camera typically has a bandpass filter configured to block other radiation, such as that projected by surgical lighting. Typically, camera 72 and projector 73 operate in a non-visible region of the spectrum, such as in the near infra-red spectrum. As is described below, at least some retroreflected radiation is typically received from marker 14, and processor 26 uses the image of the marker produced by camera 72 from the received radiation to track the marker, and thus the position and orientation of adapter 18 and of tool 16.

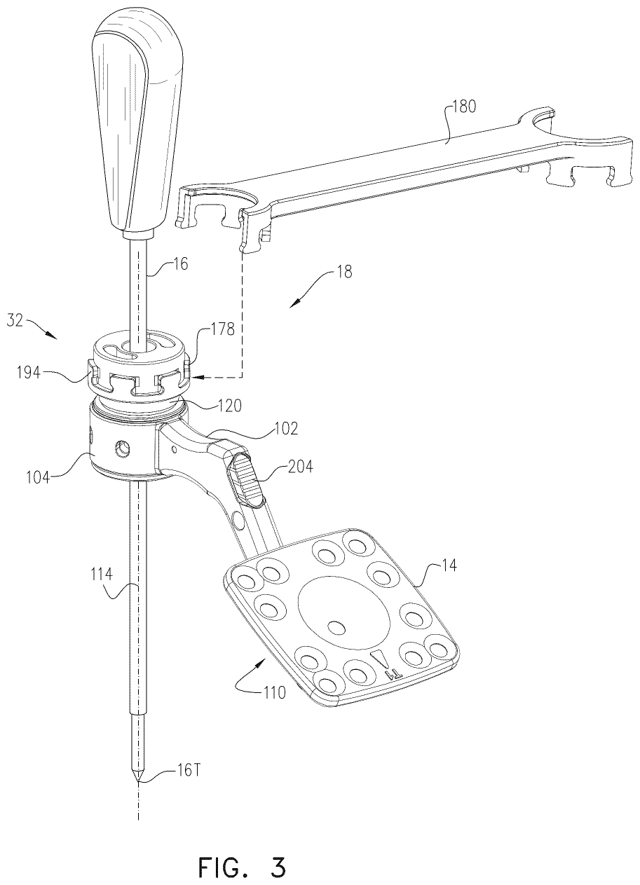

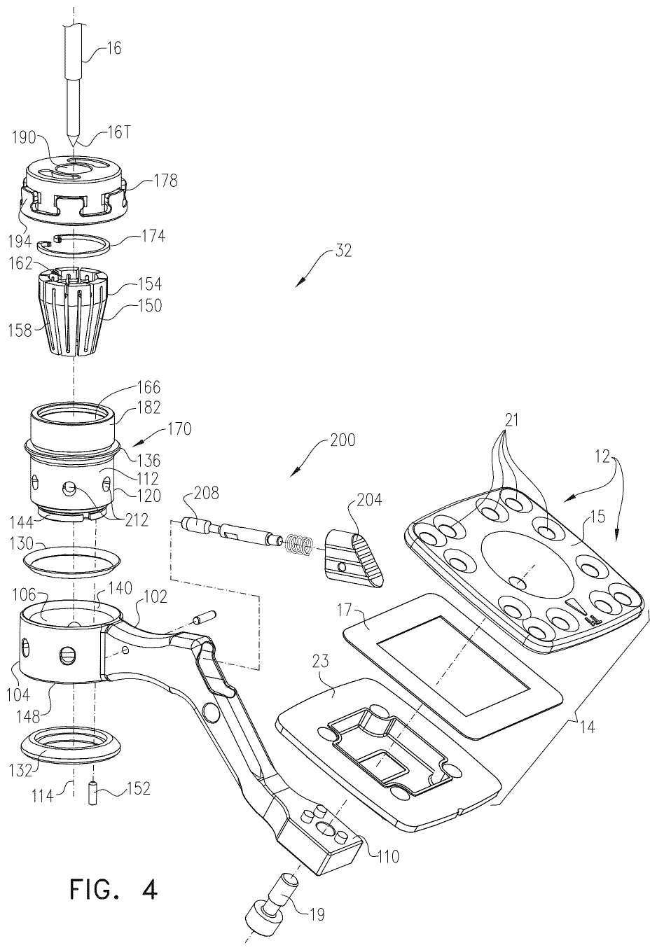

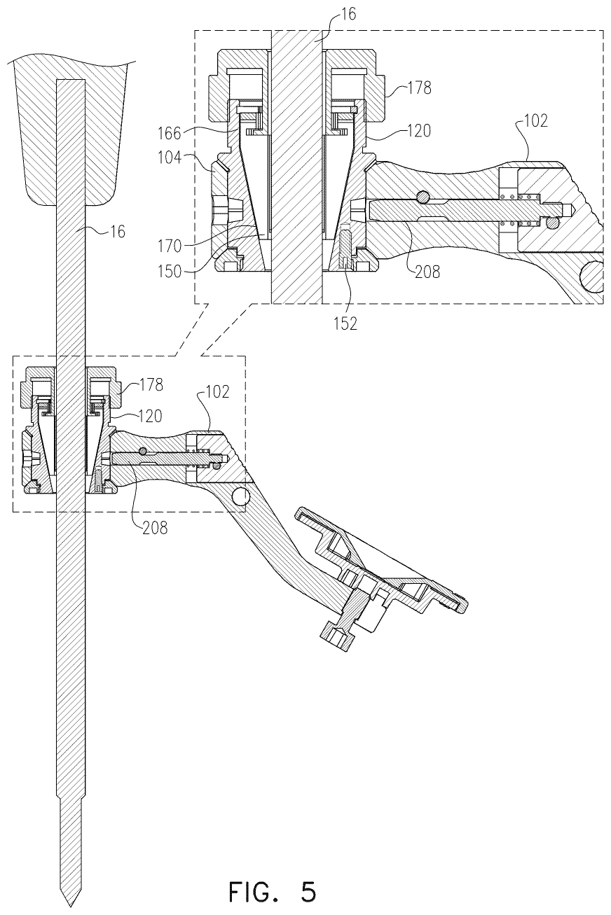

FIG. 3 is a schematic diagram illustrating tool adapter 18 and tool 16, FIG. 4 is a schematic exploded view of the tool adapter and the tool, and FIG. 5 is a schematic cross-section of the tool adapter and the tool, according to an embodiment of the present invention. Tool adapter 18 is formed from an adapter arm 102, which terminates at an upper or distal end of the arm in a circular opening 104, and which terminates at a lower or proximal end of the arm at a connection 110 which fixedly connects the arm to marker 14. A marker similar to marker 14 is described in U.S. patent application Ser. No. 16/199,281 to Messinger et al., which is incorporated herein by reference.

In a disclosed embodiment arm 102 is formed as a single unit. However, in an alternative embodiment arm 102 may be formed as two or more units fixedly connected together. For example, opening 104 may be formed as a separate unit from the remainder of arm 102, and the opening may be fixedly connected by any convenient means to the remainder of the arm.

A tool grip 32 fixedly holds tool 16, as is described in more detail below, and the tool grip is rotatingly connected to circular opening 104, so that tool 16 rotates with respect to the opening as the grip rotates.

Circular opening 104 comprises an internal cylindrical surface 106 having a diameter that is typically in a range 10 mm-50 mm, and the surface defines an axis of symmetry 114 of the opening. Tool Grip 32 comprises a rigid collet holder 120, having an external cylindrical surface 112 with a diameter slightly smaller than that of internal cylindrical surface 106, that fits into opening 104. Except as described below, collet holder 120 is able to freely rotate within opening 104.

After assembling collet holder 120 into opening 104, the holder is held, so that it cannot translate along axis 114, by an upper circular washer 130 and a lower locking nut 132. Locking nut 132 acts as a washer due to its geometry and the material from which it is formed. The washer and nut are typically formed from a low friction material such as PEEK, and act as friction bearings, preventing the holder from translating along axis 114 while allowing the holder to rotate about the axis. Upper washer 130 is held in place between a circular protruding ridge 136 fixedly formed on surface 112 and an upper edge 140 of opening 104.

Lower locking nut 132 is threaded and is configured to mate with a threaded lower region 144 of holder 120, and is dimensioned to mate with a lower edge 148 of opening 104. The lower locking nut is held in place in region 144 by adhesive between the nut and the region, and with a set screw 152 that screws into region 144. Systems other than set screw 152 for holding the locking nut in place, such as a dowel pin, will be familiar to those having ordinary skill in the art, and all such systems are assumed to be comprised within the scope of the present invention.

As is described below, in some embodiments the locking nut may be used to limit, in a controlled manner, the freedom of rotation of arm 102 around collet holder 120.

A collet 150 fits within collet holder 120. Collet 150 has an upper external portion 154 that is cylindrical, and a lower external portion 158 that is conical. Collet 150 also has a central cylindrical aperture 162 that is dimensioned to accept tool 16 when the collet is uncompressed, and to grip the tool when the collet is compressed. An embodiment of the invention comprises a set of collets, each collet of the set being able to accept tools with a range of diameters.

Collet holder 120 has an internal upper cylindrical surface 166 and a lower conical surface 170, the two internal surfaces being dimensioned to mate with the external surfaces of collet 150 (FIG. 5).

Collet 150 is held in place within the collet holder by a retaining spring 174, the retaining spring being sprung against a dedicated groove in upper cylindrical surface 166. A collet fastening head 178 is configured to screw onto a threaded outer surface 182 of collet holder 120. Fastening head 178 is configured internally to mate with spring 174, so that when the head is screwed on surface 182 so as to move towards the collet holder, it pushes on spring 174 and thus pushes and compresses collet 150.

An internal cylindrical tube 190 is fixed to an upper surface of collet fastening head 178, the tube having an internal diameter larger than the external diameter of cylindrical tool 16. An outer surface of fastening head 178 is configured to be able to be gripped by wrench 180 so that the wrench is able to turn the fastening head. By way of example, in one embodiment the outer surface has protuberances 194, and wrench 180 mates with the protuberances so that the wrench is able to rotate the fastening head.

Once tool 16 is positioned within adapter 18, so that it traverses tube 190, rotation of collet fastening head 178 pushes on a flat edge of collet 150 causing the collet to be compressed, and thus to grip tool 16. It will be appreciated that while the tool is gripped by the collet, adapter arm 102 is able to rotate around the tool.

From review of the above description, it will be appreciated that tool grip 32 comprises collet holder 120, and other elements described herein that are connected to the collet holder and collet 150, apart from arm 102 and tool 16.

In some embodiments arm 102 comprises a locking mechanism 200. Mechanism 200 comprises an operating button 204, which is configured to toggle, via a spring and a connecting rod, a pin 208 of the mechanism to mate with or disengage from one of holes 212 in collet holder surface 112. Use of the locking mechanism facilitates attachment of tool 16 to tool adapter 18, by preventing the free rotation of collet holder 120 around the center axis of the tool while wrench 180 rotates fastening head 178.

In some embodiments circular opening 104 has holes that align with holes 212. The holes in opening 104 may be used by professional 22 to align mechanism 200 with holes 212, and may also facilitate flushing of liquids and avoidance of residual liquid when adapter 18 is cleaned.

As stated above marker 14 is fixedly attached to lower connection 110. In an illustrated embodiment marker 14 comprises optical elements 12 formed on marker 14. Elements 12 may be formed as a plurality of apertures 21 in an upper marker section 15, the apertures being backed by a retroreflective sheet 17 that is held in place with respect to the apertures by a lower marker section 23 fixed to the upper marker section. Marker 14 is fixedly connected by a screw 19 to lower connection 110.

Elements 12 are typically configured to have no rotational or reflective axis of symmetry, so that processor 26 is able to use the image of marker 14, including the images of elements 12 acquired by image capturing device 68 and/or camera 72 of assembly 24, to track the marker, i.e., to determine the location and orientation of the marker in a frame of reference defined by the assembly.

In some embodiments, typically on production of adapter 18, locking nut 132 may be used to limit, in a controlled manner, the freedom of rotation of arm 102 around collet holder 120. In this case the locking nut acts as a torque limiting device. (With no limitation on its rotation, during a procedure professional 22 may inadvertently cause the arm to rotate. While, as described herein, inadvertent rotation of the arm does not affect the functioning of system 20, such rotation may be undesired.)

With no limit on the arm rotation, it will be understood that once tool 16 has been clamped by collet 150 to collet holder 120, and the tool is held horizontal, the weight of arm 102, i.e. the force on the arm due to gravity, will cause the arm to rotate about axis 114 to a vertical “6 o'clock” position. As stated above, locking nut 132 may be adjusted to counteract the force of gravity, so that when tool 16 is held horizontal, arm 102 does not rotate but is also horizontal, in a “3 o'clock” position. The locking nut acts to provide a countervailing torque to the torque generated by gravity. The countervailing torque, herein also termed a threshold torque, does not completely prevent rotation of arm 102, but reduces inadvertent rotation of the arm, since rotation only occurs when the threshold torque is exceeded.

It will be understood that the threshold torque provided by locking nut 132 prevents adapter 18 from rotating due to the force of gravity, but does not apply too much friction that could prevent smooth rotation of tool 16 by professional 22. For an arm and marker having a mass of 80 g, and a center of mass 100 mm from axis 114, the threshold torque applied to arm 192 is approximately 8 N·cm.

In addition to acting as a torque limiting device, locking nut 132 also acts as a controller of the amount of total indicated runout (TIR) of a tool gripped by adapter 18. If locking nut 132 is set to have a threshold torque of 8 N·cm, then the TIR is approximately 60 microns or less.

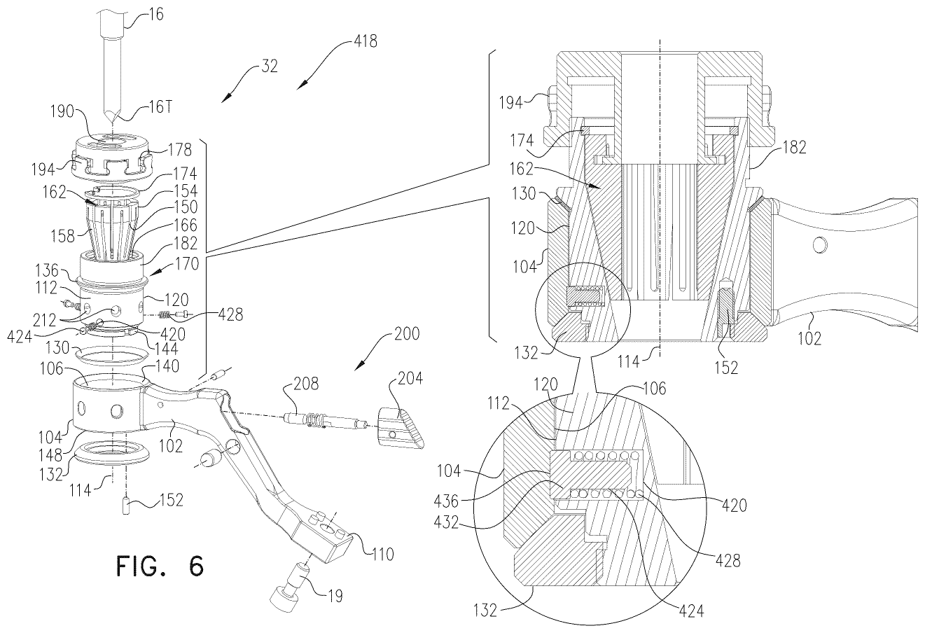

FIG. 6 is a schematic diagram illustrating a tool adapter 418 and tool 16, according to an alternative embodiment of the present invention. Apart from the differences described below, the operation of adapter 418 is generally similar to that of adapter 18 (FIGS. 1-5), and elements indicated by the same reference numerals in both adapters 18 and 418 are generally similar in construction and in operation.

In contrast to adapter 18, in adapter 418 three substantially similar blind holes 420 are formed in collet holder 120. Blind holes 420 are distributed symmetrically about axis 114, and are orthogonal to the axis.

Three substantially similar springs 428 are inserted into holes 420, and three substantially similar pins 424 are inserted into the springs. Each pin 424 has a terminal shoulder 432 which has an outer diameter that fits to the diameter of its hole 420.

In addition, the springs and pins are dimensioned so that after insertion each spring lower end contacts the base of its blind hole, the spring upper end contacts shoulder 432, and an exposed surface 436 of the shoulder slightly protrudes from surface 112, as illustrated in FIG. 6.

After assembly of adapter 418, each surface 436 pushes against internal surface 106 of opening 104 with a force that is a function of parameters of spring 428, i.e., the spring constant of the spring as well as its changed length. The pushing force in turn generates a frictional force on opening 104 when the opening rotates, or attempts to rotate, about axis 114, and the frictional force provides the threshold torque referred to above.

It will be understood that the threshold torque may be set to the value of 8 N·cm cited above, or to a value above or below this value, by selecting a spring with appropriate parameters, as well as by selecting the materials from which pin 424 and opening 104 are formed. Thus, any desired threshold torque may be implemented by these selections, without undue experimentation.

To assemble tool grip 32, springs 428 and pins 424 are first positioned in their blind holes. Opening 104 of the holder arm are then slid over collet holder 120, and over pin shoulders 432. Finally, locking nut 132 is screwed onto holder 120.

In contrast to adapter 18, wherein, as described above, locking nut 132 acts to set values of TIR and the threshold torque, in adapter 418 the two parameters can be set independently. I.e., in adapter 418 locking nut 132 is used to set the value of TIR, and springs 428 and pins 424 are used to set the value of the threshold torque.

The description above assumes that there are three sets of springs 428 and pins 424 distributed symmetrically about axis 114. However, it will be understood that any other convenient plurality of springs and pins distributed symmetrically about the axis, such as two, four, or five springs and pins, may be used as a torque limiting device.

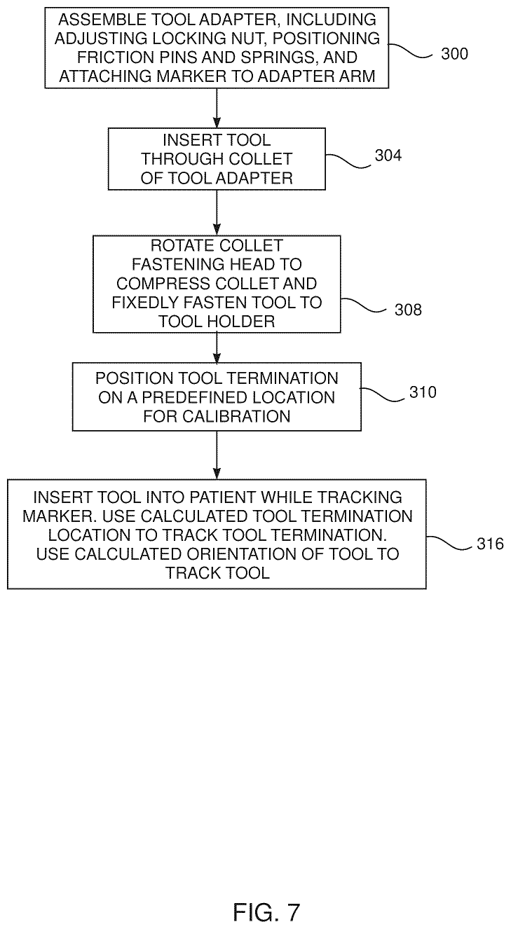

FIG. 7 is a flowchart of steps performed in using tool adapter 418 in augmented reality system 20 (FIG. 1), according to an embodiment of the present invention. Those having ordinary skill in the art will be able to adapt the description below, mutatis mutandis, for tool adapter 18.

In an initial step 300 tool adapter 418 is assembled, as described above with reference to FIGS. 3, 4, 5 and 6. The assembly typically includes adjusting locking nut 132 to provide a pre-determined TIR, and positioning pins 424 and springs 428 as described above. Also marker 14 is attached to connection 110 of arm 102.

In a tool insertion step 304, cylindrical tool 16 is inserted through aperture 162 of collet 150. Since the tool is to be used in the medical procedure referred to above on patient 30, tool 16 is inserted so that tip 16T of tool 16 is below the level of marker 14.

In a collet activation step 308, wrench 180 is used to rotate collet fastening head 178 so as to compress collet 150, the compression causing the collet to grip tool 16. While wrench 180 is being used, locking mechanism 200 should be activated to lock collet holder 120 in place, as described above, so as to facilitate the compression of collet 150.

As described above, processor 26 is able to track marker 14. Thus, in a calibration step 310, professional 22 positions tool termination 16T on a predefined location, and processor 26 acquires an image of marker 14. From the acquired image, the processor calculates a position of the marker, i.e., its location and orientation, and forms a vector correspondence between the marker position and tool termination 16T and the orientation of tool 16. I.e., the processor “learns” how to translate the marker position to tool termination 16T and to the direction of axis 114. It will be understood that the vector correspondence is unchanged if arm 102 rotates around axis 114, since the marker is fixed to the arm.

In a tool insertion step 316, tool 16 is inserted into patient 30 while processor 26 tracks marker 14. The tracking of the marker provides the processor with the location and orientation, i.e., the marker position, of the marker. From the marker position the processor is able to find, using the vector correspondence found in step 310, the location of tool termination 16T and the orientation of tool 16. The processor is able to use the orientation of tool 16 and the location of tool termination 16T in presenting correctly registered images in assembly 24.

In one embodiment tool 16 comprises a screwdriver, which is inserted into patient 30 so that professional 22 is able to adjust a pedicle screw. It will be appreciated that while the screwdriver is turned, processor 26 is still able to track the screwdriver orientation and its termination, using the vector correspondence found in calibration step 310, so long as marker 14 is tracked.

Alternatively or additionally, during the procedure positioning marker 14 may obstruct the professional's view of the patient, and/or the view of the patient as acquired by devices 68 and/or camera 72. In any of these events, professional 22 may rotate arm 102 about axis 114, applying a torque greater than the threshold torque applied in step 300, so that marker 14 no longer obstructs the view, but while processor 26 continues to track the marker. Since marker 14 continues to be tracked, the processor is able to use the new tracked location of the marker to continue tracking tool 16 and termination 16T of the tool, since the spatial relationships, i.e., the vector correspondence, between the tool and the marker, and between the tool termination and the marker, are unchanged by the tool adapter rotation about axis 114.

It will be appreciated that the embodiments described above are cited by way of example, and that the present invention is not limited to what has been particularly shown and described hereinabove. Rather, the scope of the present invention includes both combinations and subcombinations of the various features described hereinabove, as well as variations and modifications thereof which would occur to persons skilled in the art upon reading the foregoing description and which are not disclosed in the prior art.

We claim:

1. A tool adapter, comprising:
an adapter arm having a proximal end terminating with a connection and a distal end comprising a circular coupling, the circular coupling having a center and defining an axis orthogonal to the circular coupling and passing through the center; and
a tool grip rotatingly connected to the circular coupling so as to permit rotation of the tool grip about the axis, the tool grip being configured to fixedly retain a tool along the axis.

2. The tool adapter according to claim 1, wherein the tool grip comprises a collet housed in a collet holder, and wherein the collet is configured on compression to grip the tool.

3. The tool adapter according to claim 2, wherein the collet holder is held by the circular opening, and is configured to be unable to translate along the axis.

4. The tool adapter according to claim 2, and comprising a collet fastening head configured to screw onto the collet holder so as implement the compression of the collet.

5. The tool adapter according to claim 1, and comprising a position marker fixedly connected to the connection of the proximal end in a preset spatial relationship with the axis, the marker comprising optical elements enabling the marker to be tracked spatially, so that tracking of the marker provides tracking of the tool retained by the tool grip.

6. The tool adapter according to claim 1, and comprising a torque limiting device coupled to the tool grip and configured to apply a preset threshold torque to the grip so that rotation of the tool grip is permitted when the threshold torque is exceeded.

7. The tool adapter according to claim 6, wherein the torque limiting device comprises a plurality of pins retained within the tool grip and configured to exert force against the circular opening.

8. The tool adapter according to claim 1, and comprising a total indicated runout (TIR) controller coupled to the tool grip and configured to apply a pre-determined TIR to the tool.

9. A method for conducting image guided surgery, comprising:
providing an adapter arm having a proximal end terminating with a connection and a distal end comprising a circular coupling, the circular coupling having a center and defining an axis orthogonal to the circular coupling and passing through the center;
rotatingly connecting a tool grip to the circular coupling so as to permit rotation of the tool grip about the axis, the tool grip being configured to fixedly retain a tool along the axis;
fixedly connecting a position marker to the connection of the proximal end of the adapter arm in a preset spatial relationship with the axis, the position marker comprising optical elements enabling the marker to be tracked spatially, so that tracking of the position marker provides tracking of the tool fixed to the enclosure; and
presenting an image of the tracked tool to a professional performing the surgery.

10. The method according to claim 9, wherein the tool grip comprises a collet housed in a collet holder, and wherein the collet is configured on compression to grip the tool.

11. The method according to claim 10, wherein the collet holder is held by the circular opening, and is configured to be unable to translate along the axis.

12. The method according to claim 10, and comprising configuring a collet fastening head to screw onto the collet holder so as implement the compression of the collet.

13. The method according to claim 9, and comprising coupling a torque limiting device to the tool grip and configuring the device to apply a preset threshold torque to the grip so that rotation of the tool grip is permitted when the threshold torque is exceeded.

14. The method according to claim 13, wherein the torque limiting device comprises a plurality of pins retained within the tool grip and configured to exert force against the circular opening.

15. The method according to claim 9, and comprising coupling a total indicated runout (TIR) controller to the tool grip and configuring the controller to apply a pre-determined TIR to the tool.
